(19) United States
(12) Patent Application Publication
(10) Pub. No.: US 2025/0049491 A1
(43) Pub. Date: Feb. 13, 2025

(54) DEVICE FOR VAGINAL OR RECTAL CRYOTHERAPY, AND METHODS OF PRODUCTION AND USE

(71) Applicants: Barbara Ann Dodson, Cushing, OK (US); Alfredo Salazar, Tulsa, OK (US); Jane S. Salazar, Mission, TX (US); Donald J. Kyle, Las Vegas, NV (US)

(72) Inventors: Barbara Ann Dodson, Cushing, OK (US); Alfredo Salazar, Tulsa, OK (US); Jane S. Salazar, Mission, TX (US); Donald J. Kyle, Las Vegas, NV (US)

(21) Appl. No.: 18/796,984

(22) Filed: Aug. 7, 2024

Related U.S. Application Data
(60) Provisional application No. 63/531,366, filed on Aug. 8, 2023.

(51) Int. Cl. A61B 18/02

(52) U.S. Cl. CPC A61B 18/02; A61B 2018/0212

(57) ABSTRACT

An insert, and methods of production and use, for vaginal or rectal cryotherapy to relieve chronic pelvic pain, inflammation, edema, pain related to pelvic floor muscle cramping and spasms, vaginal pain, bladder pain, rectal pain, and other pain and symptoms.

### RELATED CASE

This application claims the benefit of U.S. Provisional Patent Application No. 63/531,366 filed Aug. 8, 2023 and incorporates said provisional application herein by reference in its entirety as if fully set forth at this point.

### FIELD OF THE INVENTION

The present invention relates to devices and methods for vaginal cryotherapy treatment of Myofascial Pain Syndrome in the Pelvic Floor (commonly referred to as Chronic Pelvic Pain) to relieve inflammation, edema, and pain related, e.g., to pelvic floor muscle cramping and spasms. The present invention also relates to devices and methods for vaginal, rectal, or other cryotherapy treatment of vaginal pain, bladder pain, rectal pain, etc. resulting from other causes. In addition, the present invention further relates to methods and equipment for producing the cryogenic treatment devices.

### BACKGROUND OF THE INVENTION

Pelvic floor pain is suffered by millions of women around the world. It degrades the quality of life of these women and the cost of treatment for relieving the pain can be quite high. Pelvic floor pain is a symptom which can result from numerous different causes and diseases including, but not limited to, pelvic floor dysfunction, interstitial cystitis (painful bladder syndrome), pudendal neuralgia, vulvodynia, uterine fibroids, endometriosis, and irritable bowel syndrome.

The pelvic floor muscles are arranged to form complex layers that support the pelvic organs, uterus, and span the bottom of the pelvis. These muscles are innervated by sacral nerves projecting from the sacrum, notably S2, S3, and S4.During a Chronic Pelvic Pain (CPP) episode, painful lumps or nodules often develop in these muscles (myofascial trigger points) together with inflammation leading to muscle spasms, cramping, and pain. The etiology is complex, having both neurological and musculoskeletal components. As episode of CPP can arise without warning and can interfere with lifestyle events including work, recreation, and exercise.

It is estimated that over 20% of all women will suffer from pelvic floor pain at some time in their lives, typically between the ages 18 and 50, and that the pain will commonly last for up to six months or more. In a great number cases the pain lasts significantly longer, with many women suffering chronically for most of their lives.

CPP is often misdiagnosed by physicians and only an estimated 33% of afflicted women ever report their condition such that its prevalence is under-represented. Although there are a number of treatment methods commonly applied including medication, physical therapy, nerve block, and massage, there is also a very high failure rate. Taken together, CPP is under-reported by women, often misdiagnosed by physicians, and treatment options are often not sufficient to manage the pain, making this condition a large unmet need in women's health.

Currently, the most common non-invasive treatments for pelvic floor pain include expensive pelvic floor physical therapy, and the use of strong prescription pain medications, such as hydrocodone, tramadol, vaginal valium, or belladonna suppositories, which are often addictive and/or somewhat dangerous.

Invasive treatments for pelvic floor pain, which are less commonly used, include: INTERSTIM implants in the lower back which emit mild electrical pulses to stimulate the sacral nerves; cystoscopy with bladder hydrodistension; and BOTOX injections. In addition to being invasive and expensive, these treatments do not work for all women and can sometimes make the pain worse.

Many men also experience pain in the pelvis at some point in their life with symptoms including difficult, painful, or frequent urination, and pain in the area of the bladder, groin, anus, and abdomen. In many cases this pain is referred to as “prostatitis”, meaning inflammation of the prostate. A more accurate terminology is widely used for Chronic Pelvic Pain Syndrome (CPPS) in men and includes CCPS I and CCPS II if a bacterial infection is present, and CCPS III and CCPS IV if no bacterial infection is present. Current front-line therapy for men generally includes an extended course of oral antibiotics (for CCPS I and CCPS II), physical therapy, muscle relaxants and other medications, and/or hospitalization.

Consequently, a need exists for a treatment which is highly effective for relieving CPP, CPPS, and other pelvic floor pain. A need particularly exists for a treatment which (a) is non-invasive, (b) does not involve the use of narcotic drugs or other medications, (c) is very low cost and affordable, and (d) can be easily used and performed by the patient at home to relieve pain whenever needed.

### SUMMARY OF THE INVENTION

The present invention provides a cryotherapy device and method, for vaginal or rectal use, which satisfy the needs and alleviate the problems discussed above. The inventive cryotherapy device, which can be approximately the same size as a women's tampon, is sanitary and disposable, and the patient can easily insert the inventive device into the vagina or rectum by finger. There is no need to purchase or use an applicator. After inserting the inventive device, the patient can resume their normal activities. Then, after about ten minutes or whenever desired, the device can be easily removed and disposed of.

The inventive vaginal or rectal cryotherapy device greatly relieves pain by reducing blood flow to situs of the pain, which significantly reduces inflammation and swelling (edema). And the inventive device and treatment further relieve the pain experienced by the patient by reducing nerve activity in the problem area.

The inventive cryotherapy device and treatment are effective for improving, managing, preventing, and/or eliminating pain, cramps, and spasms of the pelvic floor muscles. The inventive device and treatment method can also be used to treat and relieve bladder incontinence, fecal incontinence, bladder retention, and constant bladder urgency.

The inventive device and method are designed as a non-pharmacological over-the-counter treatment for CPP to be used alone or in combination with other known therapies. The device and method uniquely allow for the application of cryotherapy in close proximity to the myofascial trigger points, thus leveraging the known benefits of cryotherapy to reduce muscle spasm and cramping, reduce inflammation, and attenuate pain-producing nerve impulses. The device is also discrete when used, and makes daily life a possibility while in use. Practically, the inventive device is easily portable and inexpensive to obtain, and without a prescription. The inventive device and method will empower women to gain some control over the management of their CPP episodes

In one aspect, there is provided a cryotherapy device and method which preferably comprise or use a frozen or near-frozen vaginal insert. The vaginal insert preferably comprises a cover having a fluid therein which is frozen prior to use/insertion. The inventive device also preferably comprises a string or ribbon which is tied around or otherwise attached to the cover for removing the inventive cryogenic treatment device, in the same manner, e.g., as a tampon, after the treatment or whenever desired by the user.

In another aspect, there is provided an insert for vaginal or rectal cryogenic treatment. The insert preferably comprises: (i) a longitudinally extending container having a longitudinal axis, an interior, and a closed forward longitudinal end; (ii) a thermal material, for chilling and/or freezing, which is placed in the interior of the longitudinally extending container via a rearward longitudinal end of the longitudinally extending container; and (iii) a closure on the rearward longitudinal end of the longitudinally extending container which retains the thermal material in the interior of the longitudinally extending container.

In another aspect, the insert can further comprise (i) the longitudinally extending container being a molded container and (ii) the closure comprising a cap which is molded on the rearward longitudinal end after the thermal fluid is placed in the longitudinally extending container.

In another aspect, the insert can further comprise one or more structural ribs in the interior of the longitudinally extending container which help the longitudinally extending container to retain its shape for insertion.

In another aspect, the insert can further comprise the one or more structural ribs being one or more radially extending partitions in the interior of the longitudinally extending container which divide the interior of the longitudinally extending container into two or more separate, longitudinally extending chambers for the thermal material.

In another aspect, there is provided a method of cryogenic treatment. The method of cryogenic treatment preferably comprises the steps of: (a) chilling an insert of the type described above until the thermal material is frozen and/or reaches a temperature equal to or below 10° C.; (b) after step (a), applying a hygienic lubricant to the insert; and then (c) inserting the insert into a vaginal opening or a rectal opening by finger without using an applicator.

In another aspect, there is provided a method of forming an insert for vaginal or rectal cryogenic treatment. The method of forming the insert for vaginal or rectal cryogenic treatment preferably comprises the steps of: (a) inserting an inner molding element into an outer molding element; (b) injecting a molding material into the outer molding element to form a molded, longitudinally extending container having a longitudinal axis, an interior, a closed forward longitudinal end, and one or more radially extending partitions in the interior of the longitudinally extending container which divide the interior of the longitudinally extending container into two or more separate, longitudinally extending chambers having rearward openings at a rearward longitudinal end of the longitudinally extending container; (c) removing the inner molding element from the outer molding element; (d) delivering a thermal material, for chilling and/or freezing, through the rearward openings into the two or more separate, longitudinally extending chambers; (e) placing a cap molding element on the outer molding element; and then (f) injecting a molding material into the cap molding element to form a molded cap on the rearward longitudinal end of the longitudinally extending container.

The inner molding element used in this method is preferably configured to form the one or more radially extending partitions and the longitudinally extending chambers in the interior of the longitudinally extending container in step (b). The molded cap closes the rearward openings of the two or more separate, longitudinally extending interior chambers of the longitudinally extending container.

Further objects, features, and advantages of the present invention will be apparent to those in the art upon examination of the accompanying drawings and upon reading the following Detailed Description of the Preferred Embodiments.

### BRIEF DESCRIPTION OF THE DRAWINGS

FIG. 1 is a perspective view of an embodiment 2 of the cryotherapy insert provided by the present invention.

FIG. 2 is a side view of an embodiment 20 of the cryotherapy insert provided by the present invention.

FIG. 3 is a cutaway end view of the inventive cryotherapy insert 20 as seen from perspective 3-3 shown in FIG. 2.

FIG. 4 is an elevational side view of an outer molding element 50 for producing the inventive cryotherapy insert 20.

FIG. 5 is a top view of the outer molding element 50.

FIG. 6 is a side view of an inner molding element 52 for producing the inventive cryotherapy insert 20.

FIG. 7 is a forward end view of the inner molding element 52.

FIG. 8 is a rearward end view of the inner molding element 52.

FIG. 9 is a perspective view of a cap molding element 54 for producing the inventive cryotherapy insert 20.

FIG. 10 is a side view of the cap molding element 54.

FIG. 11 is a top view of the cap molding element 54.

### DETAILED DESCRIPTION OF THE PREFERRED EMBODIMENTS

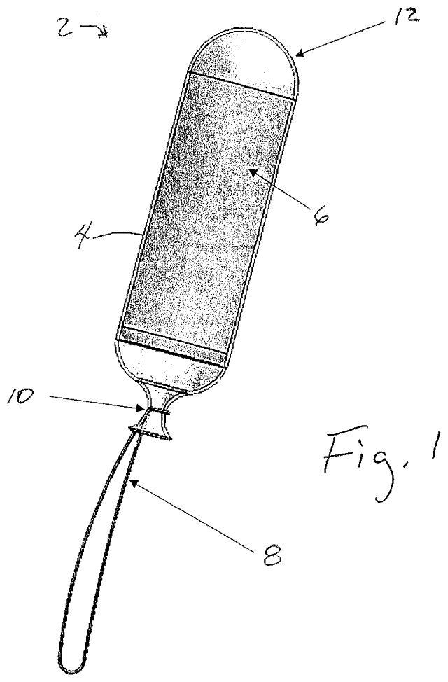

An embodiment 2 of the inventive vaginal or rectal cryotherapy device is illustrated in FIG. 1. As shown in FIG. 1, the inventive cryotherapy insert 2 preferably has a size and shape similar to a woman's tampon (i.e., has a tampon shape) and preferably comprises: (i) a container 4 which is a cover having a closed distal end 12; (ii) a thermal material such as a fluid 6 contained in the cover 4 which is frozen or chilled to a near frozen state prior to use; and (iii) a string or ribbon 8 (preferably a nitrile string) which is tied around the proximal end 10 of, or is otherwise secured to, the cover 4.

After filling the cover 4 with the fluid 6, the proximal end 10 of the cover 4 can be sealed by heat sealing and/or tying with the string or ribbon 8 (or by any other suitable means) to retain the fluid 6 in the cover 4 and prevent leakage. If included, the string or ribbon 8 will also facilitate the removal of the inventive insert 2 after use.

The cover 4 can be substantially any type of cover which is safe to use in the body, is of the size and shape desired, is compatible with and effective for containing and freezing or substantially freezing the fluid 6, and readily permits heat transfer between the body and the frozen or near frozen fluid 6 within the cover 4. The cover 4 will preferably be latex-free and will more preferably be a nitrile sleeve having generally the same degree of flexibility as a finger of a nitrile glove. Alternatively, the cover 4 could be a flexible sleeve formed of silicone or other flexible materials, or could be a more rigid, or non-flexible, or substantially non-flexible tube formed of a suitable plastic material.

The cover 4 will preferably be a flexible nitrile cover, as discussed above, and the fluid 6 contained in the cover 4 will preferably be a solution which will freeze to a rigid, solid state to facilitate insertion of the inventive cryotherapy device 2 without an applicator.

The fluid 6 contained in the cover 4 can be any fluid which (a) is safe for use, (b) will not harm the patient should a leak occur, and (c) can preferably be chilled to a solid frozen state, or a near or sufficiently frozen state, in a typical home freezer, such that the combined rigidity of the chilled fluid 6 and the cover 4 will allow the inventive device 2 to by inserted by finger, without the use of an applicator.

Examples of fluids 6 suitable for use in the inventive method and device 2 include, but are not limited to, saline solutions, distilled water, mixtures of distilled water and alcohol, and mixtures of salt and distilled water. The fluid 6 used in a nitrile or other flexible cover 4 will preferably be distilled water or a saline solution which will freeze solid in a home freezer. A saline solution, if used, will preferably comprise, or consist entirely of, sodium chloride and water (preferably distilled water), wherein the sodium chloride concentration will most preferably be about 0.9 g (i.e., 0.9 g±10%) of sodium chloride per 100 ml of solution.

To facilitate insertion, the cover 6 of the inventive cryotherapy device 2 will also preferably be covered with a vaginal jelly, gel, or other lubricant material.

The inventive cryotherapy device 2 will typically be provided to the user in a non-frozen state. Then, in accordance with the method of the present invention, the user will place the inventive device 2 in their freezer for approximately one hour, or as long as needed to achieve and maintain the solid or near solid frozen/chilled state desired. Next, the user will (i) insert the inventive device 2 into the vagina or rectum, preferably by finger without the use of an applicator, and then (ii) allow the device 2 to remain in the vagina or rectum until the fluid 6 is melted or as long as otherwise desired. Then, the user will preferably use the string or ribbon 8 to remove the cryotherapy device 2 and will dispose of the device 2 by tossing into a waste bin.

By way of example, but not by way of limitation, the inventive vaginal cryotherapy device 2 can be provided to the user in a blister pack, preferably a blister pack containing multiple disposable devices 2, so that the device or devices 2 can be removed from the blister pack 2 before or after, more preferably after, freezing.

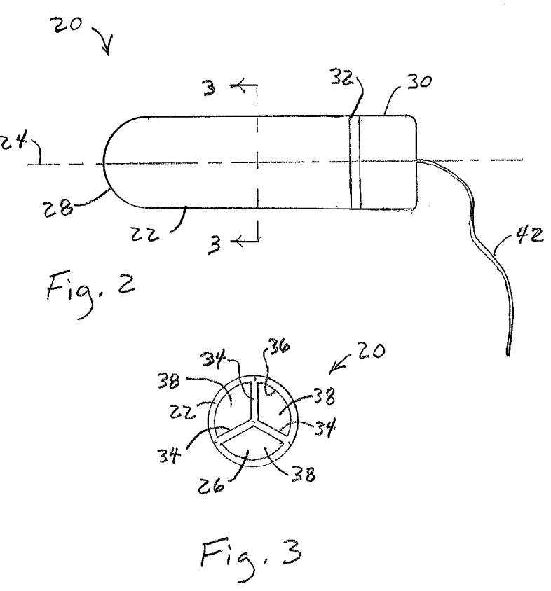

An alternative embodiment 20 of the inventive cryotherapy insert for vaginal or rectal use is illustrated in FIGS. 2 and 3. The inventive insert 20 preferably comprises: (i) a longitudinally extending container 22 having a longitudinal axis 24, an interior 26, and a closed forward longitudinal end 28; (ii) a thermal material, for chilling and/or freezing, which is contained in the interior 26 of the longitudinally extending container 22; and (iii) a closure 30 on the rearward longitudinal end 32 of the longitudinally extending container 22 which retains the thermal material in the interior 26 of the container 22.

The inventive insert 20 preferably also includes one or more ribs 34 formed in the interior 26 of the longitudinally extending container 22 which provide structural strength and help the container 22 to retain its shape during insertion. The one or more ribs 34 can be of any size, shape, or orientation (including even a single rib in a helical configuration) but will preferably be radial ribs which (a) extend radially from the interior wall 36 of the container 22 to or through the longitudinal axis 24 and also (b) extend longitudinally in the interior 26 of the longitudinally extending container 22. The one or more ribs 34 will most preferably be in the form of radially extending partitions in the interior 26 of the longitudinally extending container 22 which divide the container 22 into two or more separate, longitudinally extending chambers 38 for receiving and holding the thermal fluid. The longitudinally extending container 22 will preferably have from two to four such chambers 38 of equal size formed therein.

The container 22 will more preferably have three separate, equally sized, longitudinally extending chambers 38 formed in the interior 26 thereof. The three chambers 38 will preferably be formed by three equally spaced rib partitions 34 which extend radially from the longitudinal axis 24 to the interior wall 36 of the longitudinally extending container 22.

The longitudinally extending container 22 and the one or more ribs 34 are preferably formed by molding such that the interior ribs 34 are integrally molded and formed with the longitudinally extending container 22. Prior to placing the closure 30 on the rearward longitudinal end 32 of the longitudinally extending container 22, the rearward ends of the interior chambers 38 of the container 22 will be open so that the thermal material can be injected or otherwise placed in the interior chambers 38 through the open rearward ends thereof.

The material used for molding the longitudinally extending container 22 and the interior ribs 34 can generally be any material which is hygienically safe, provides sufficient strength for insertion and use, and has satisfactory heat transfer properties for readily transferring heat from the body to the chilled and/or frozen thermal material in the container 22. The molded insert 20 can be formed of a rigid material or can be formed of a material which provides any desired degree of flexibility. If a flexible material is used, the degree of flexibility will preferably still be such that, after freezing or chilling the thermal material inside the inventive insert 2, the insert 2 will be sufficiently rigid or firm to permit the insert 2 to be inserted into the vaginal or rectal opening by hand without the use of an applicator.

Although many different materials having varying degrees of flexibility can be used to form the container 22 and the interior ribs 34, the material will preferably be a silicon-containing molding material and will most preferably be a de-gassed liquid silicon material. An example of a commercially available de-gassed liquid silicon material which is well-suited for use in the present invention is Ecoflex™ platinum-catalyzed silicon available from Smooth-On, Inc.

To provide sufficient structural integrity and a desirable rate of heat transfer, the thickness of the surrounding wall of the longitudinally extending container 22 and the thickness of each of the rib partitions 34 therein will preferably be in the range of from 0.5 mm to 2.5 mm. The thickness of the surrounding wall of the container 22 and the thickness of each of the interior rib partitions 34 will preferably be about 1.5 mm. The term “about”, as used herein and in the claims when referring to lengths, widths, heights, or thicknesses, means within a range of ±10%.

After the thermal fluid is placed in the interior chambers 38 of the container 22, the thermal fluid is retained in the chambers 38 by placing the closure 30 of the inventive insert 20 on the rearward longitudinal end 32 of the container 22. The rearward closure 30 of the inventive insert 20 can generally be any type of closure which is safe for use and is effective for closing the rearward openings of the interior chambers 38. The rearward closure 30 will preferably be a cap which is molded onto the rearward end 32 of the longitudinally extending container 22 after the thermal material is added to the interior chambers 38. The molded cap 30 will also preferably have a string, ribbon, cord, or thread 42 extending therefrom for removing the inventive insert 20 after use.

The inventive insert 20 can generally have any type of tampon shape. As illustrated in FIG. 2, the inventive insert 20 will most preferably comprise a cylindrical body having a closed forward end 28 which is outwardly curved. The outwardly curved forward end 28 of the insert 20 will assist with the insertion of the device 20 and will most preferably have a hemispherical or ovoidal shape.

Examples of thermal materials that are suitable for use in the inventive insert 20 for freezing and/or chilling include, but are not limited to, any of the fluids 6 discussed above. Further examples of thermal materials suitable for use in the inventive inserts 20 and 2 include polymers and aqueous blends which preferably have specific heat capacities which are similar to or higher than ice or water.

As another alternative, the thermal material used in the inventive insert 20 or 2 can comprise two separated binary components of the type used, e.g., in so-called instant ice packs for sprains and other external treatments. Instant ice packs typically utilize water and a reactant such as ammonium nitrate, calcium ammonium nitrate, or urea. This alternative eliminates the need to chill the insert 20 or 2 in a freezer or other unit prior to use—thus providing quicker, on-demand treatment and making the inventive device 20 or 2 more convenient for travel and for emergency use.

Each of the two binary components will preferably be placed in each of the interior chambers 38 of the insert 20 in a separated form wherein, e.g., at least one of the two materials will be contained in a burstable package. In addition, in this embodiment, the longitudinally extending container 22 of the inventive insert 20 will preferably be sufficiently flexible to allow the patient to burst the internal package by twisting or squeezing so that binary materials are allowed to mix and react in the insert 20 for chilling.

In the method of cryogenic treatment provided by the present invention using the inventive insert 20, the insert 20 will preferably first be chilled until the thermal material within the insert 20 is frozen and/or reaches a temperature equal to or less than 10° C. (more preferably equal to or less than: 5° C., 0° C., or −2° C.). The insert 20 can be chilled in a home freezer or other chilling unit or, if the insert 20 contains binary instant ice pack components, the insert 20 can be twisted or squeezed to cause the binary components to mix and react.

Next, for greater comfort and ease of insertion, a vaginal jelly, gel, or other hygienic lubricant will preferably be applied to the chilled insert 20.

The chilled insert 20 is then inserted into a vaginal opening or rectal opening for treatment. Although an applicator can be used to insert the chilled cryotherapy device 20 if desired, the chilled device 20 will be sufficiently firm that the patient can insert the device 20 by finger without having to purchase and use an applicator.

The inventive insert 20 can be allowed remain in the vagina or rectum until the thermal material melts or as long as otherwise desired. The user will preferably use the string, cord, thread, or ribbon 42 to remove the cryotherapy device 20 and will then dispose of the device 20 by tossing into a waste bin.

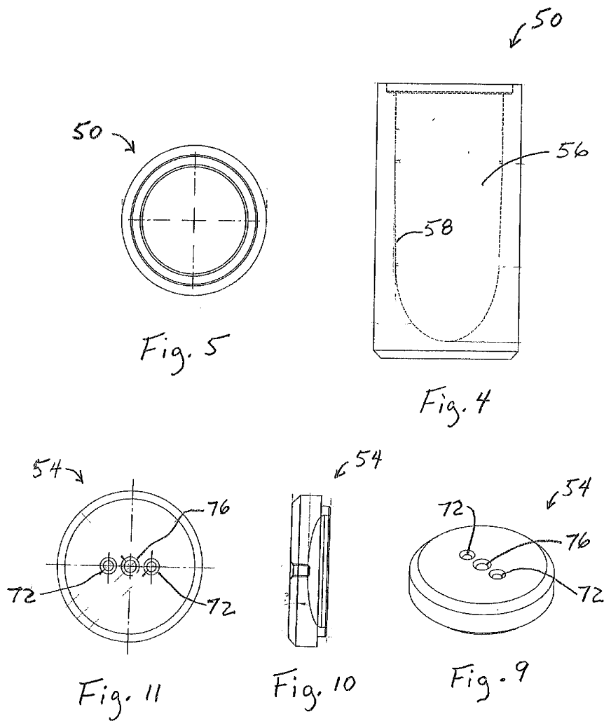

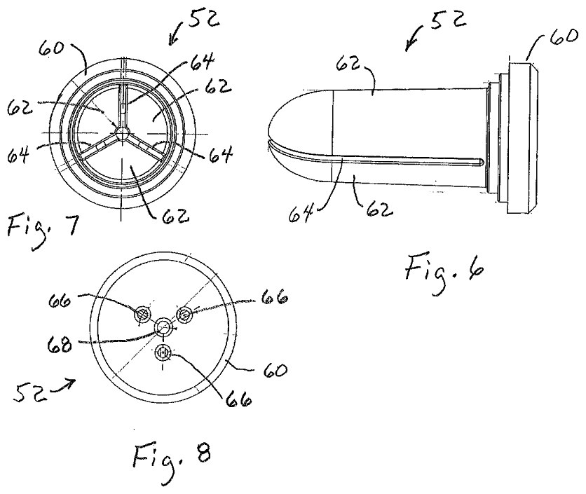

By way of example, but not by way of limitation, a set of molding elements which can be used to form the inventive insert 20 is illustrated in FIGS. 4-11. The set of molding elements includes: an outer molding element 50; an inner molding element 52; and a cap molding element 54. The outer molding element 50 has a longitudinally extending cavity 56 formed therein with an interior surface 58 which corresponds to and forms the molded exterior of (i) the longitudinally extending container 22 of the insert 20 and (ii) the closed, and preferably curved, forward longitudinal end 28 of the longitudinally extending container 22.

The inner molding element 52 preferably comprises: a flange 60 on the rearward longitudinal end of the inner molding element 52; two or more (preferably three) separate, longitudinally extending lobes 62 which extend forwardly from the rearward flange 60 of the inner molding element 52; longitudinally extending radial slots 64 which are formed between and separate the longitudinally extending lobes 62; and one or more injection openings 66, 68 which extend through the rearward flange 60.

The molding material for the inventive insert 20 will be injected into the one or more injection openings 66, 68 of the inner molding element 52 such that the molding material will (a) fill the cavity 56 of outer molding element 50 outside of and surrounding the longitudinally extending lobes 62 to form the outer wall of the longitudinally extended container 22 of the insert 20 and (b) fill the slots 64 between the longitudinally extending lobes 62 to form the rib partitions 34 in the interior of the container 22. As a result, the longitudinally extending lobes 62 create voids in the in the interior of container 22 which form the interior chambers 38 of the container 22 for receiving the thermal material.

The inner molding element 52 illustrated in FIGS. 6-8 has three longitudinally extending lobes 62 for forming three evenly spaced chambers 38 of equal size in the interior of the longitudinally extending container 22 of the inventive insert 20. The inner molding element 52 illustrated in FIGS. 6-8 also has (a) three separate injection openings 66 through the flange 60 for injecting the molding material into each of the three rib forming slots 64 which separate the lobes 62 and (b) a centered injection opening 68 provided through the flange 60 for injecting the molding material between the inner ends of the lobes 62 along what will be the longitudinal axis 24 of the longitudinally extending container 22.

The molding elements 50, 52, and 54 can be formed of stainless steel, aluminum, or any other suitable material.

In the method of the present invention for forming the inventive insert 20, the two or more lobes 62 of the inner molding element 52 are inserted into the cavity 56 of the outer molding element 50 such that the rearward flange 60 of the inner molding element 52 contacts and is stopped by the rearward end of the outer molding element 50. The molding material is then injected into the cavity 56 of the outer molding element 50 via the injection openings 66, 68 of the inner molding element 52 to form the longitudinally extending container 22 of the inventive insert having (i) a longitudinal axis 24, (ii) an interior 26, (iii) a closed forward longitudinal end 28, and (iv) one or more radially extending rib partitions 34 in the interior 26 of the container 22 which divide the interior 26 of the container 22 into two or more separate, longitudinally extending chambers 38 for receiving the thermal material.

Next, the inner molding element 52 is removed from the outer molding element 50 so that the molded, longitudinally extending container 22 of the insert 20 remains in the outer molding element 50 and the rearward longitudinal ends 70 of the interior chambers 38 are open. The thermal material used in the inventive insert 20 for chilling and/or freezing is then delivered, through the rearward openings 70, into each of the separate chambers 38 of the container 22.

After filling the interior chambers 38 with the thermal material, the cap molding element 54 is placed on the rearward end of the outer molding element 50 and the same or a different molding material is then injected through one or more injection openings 72 of the cap molding element 54. The molding material injected into the cap molding element 54 forms a molded cap 30 on the rearward longitudinal end 32 of the longitudinally extending container 22 which closes and seals the rearward openings of the interior chambers 38. Prior to injecting the cap molding material, the end of a ribbon, string, cord, or thread 42 is preferably threaded through a central opening 76 of the cap molding element 54 such that the ribbon, string, cord, or thread 42 will be anchored to, and will extend from, the molded cap 30 of the inventive insert 20.

Thus, the present invention is well adapted to carry out the objects and attain the ends and advantages mentioned above as well as those inherent therein. While presently preferred embodiments have been described for purposes of this disclosure, numerous changes and modifications will be apparent to those in the art. Such changes and modifications are encompassed within this invention as set forth in the claims.

What is claimed is:

1. An insert for vaginal or rectal cryogenic treatment, the insert comprising:
a longitudinally extending container having a longitudinal axis, an interior, and a closed forward longitudinal end;
a thermal material, for chilling and/or freezing, which is placed in the interior of the longitudinally extending container via a rearward longitudinal end of the longitudinally extending container; and
a closure on the rearward longitudinal end of the longitudinally extending container which retains the thermal material in the interior of the longitudinally extending container.

2. The insert of claim 1 further comprising the closure comprising a string, ribbon, cord, or thread which closes the rearward longitudinal end.

3. The insert of claim 1 further comprising the closure comprising a heat seal.

4. The insert of claim 1 further comprising:
the longitudinally extending container being a molded container and
the closure comprising a cap which is molded on the rearward longitudinal end after the thermal fluid is placed in the longitudinally extending container.

5. The insert of claim 4 further comprising a string, ribbon, cord, or thread which extends from the cap which is molded on the rearward longitudinal end of the container.

6. The insert of claim 1 further comprising the insert having a tampon shape.

7. The insert of claim 1 further comprising:
the longitudinally extending container comprising a cylindrical body and
the closed forward longitudinal end having an outwardly curved shape.

8. The insert of claim 7 further comprising the closed forward longitudinal end having hemispherical or ovoidal shape.

9. The insert of claim 1 further comprising one or more structural ribs in the interior of the longitudinally extending container which help the longitudinally extending container to retain its shape for insertion.

10. The insert of claim 9 further comprising the one or more structural ribs extending longitudinally in the interior of the longitudinally extending.

11. The insert of claim 9 further comprising the one or more structural ribs being one or more radially extending partitions in the interior of the longitudinally extending container which divide the interior of the longitudinally extending container into two or more separate, longitudinally extending chambers for the thermal material.

12. The insert of claim 11 further comprising:
the longitudinally extending container being a molded container and
the one or more radially extending partitions being integrally molded with and as an integral part of the longitudinally extending container.

13. The insert of claim 12 further comprising the longitudinally extending container and the one or more radially extending partitions being molded from a flexible silicone material.

14. The insert of claim 13 further comprising (i) the longitudinally extending container having a container wall and (ii) the container wall and each of the one or more radially extending partitions having a thickness in a range of from 0.5 mm to 2.5 mm.

15. The insert of claim 14 further comprising the thickness of the container wall and the thickness of each of the one or more radially extending partitions being about 1.5 mm.

16. The insert of claim 11 further comprising the one or more radially extending partitions being three radially extending walls in the interior of the longitudinally extending container which (i) extend radially outward from the longitudinally axis of the longitudinally extending container to an interior wall of the longitudinally extending container and (ii) divide the interior of the longitudinally extending container into three of said separate, longitudinally extending chambers for the thermal material.

17. The insert of claim 1 further comprising:
the longitudinally extending container being formed of a flexible material;
the thermal material which is placed in the interior of the longitudinally extending container being in a form of two binary instant ice pack components; and
at least one of the two binary instant ice pack components being held in a burstable package which separates the two binary ice pack components until use.

18. A method of cryogenic treatment comprising steps of:
a) chilling the insert of claim 1 until the thermal material is frozen and/or reaches a temperature equal to or below 10° C.;
b) after step (a), applying a hygienic lubricant to the insert; and then
c) inserting the insert into a vaginal opening or a rectal opening by finger without using an applicator.

19. A method of forming an insert for vaginal or rectal cryogenic treatment, the method comprising steps of:
a) inserting an inner molding element into an outer molding element;
b) injecting a molding material into the outer molding element to form a molded, longitudinally extending container having
a longitudinal axis,
an interior,
a closed forward longitudinal end, and
one or more radially extending partitions in the interior of the longitudinally extending container which divide the interior of the longitudinally extending container into two or more separate, longitudinally extending chambers having rearward openings at a rearward longitudinal end of the longitudinally extending container, the inner molding element being configured to form the one or more radially extending partitions and the longitudinally extending chambers in the interior of the longitudinally extending container;
c) removing the inner molding element from the outer molding element;
d) delivering a thermal material, for chilling and/or freezing, through the rearward openings into the two or more separate, longitudinally extending chambers;
e) placing a cap molding element on the outer molding element; and then
f) injecting a molding material into the cap molding element to form a molded cap on the rearward longitudinal end of the longitudinally extending container which closes the rearward openings of the two or more separate, longitudinally extending chambers of the longitudinally extending container.
